(19) United States
(12) Patent Application Publication
(10) Pub. No.: US 2023/0257263 A1
(43) Pub. Date: Aug. 17, 2023

(54) RENEWABLE ENERGY HYDROCARBON PROCESSING METHOD AND PLANT

(71) Applicant: Woodside Energy Technologies Pty Ltd, Perth (AU)

(72) Inventors: Solomon FAKA, Perth (AU); Lourens JACOBS, Perth (AU); Owen DAVIS, Perth (AU)

(21) Appl. No.: 17/961,875

(22) Filed: Oct. 7, 2022

Related U.S. Application Data
(63) Continuation of application No. PCT/AU2021/050329, filed on Apr. 9, 2021.

(30) Foreign Application Priority Data
Apr. 9, 2020 (AU) 2020901146
Oct. 5, 2020 (AU) 2020903596

(51) Int. Cl. C01B 3/38; B01J 19/24; B01J 19/00; F28D 21/00; C25B 1/04; C25B 9/65; C25B 15/08

(52) U.S. Cl. CPC C01B 3/384; C01B 3/388; B01J 19/2415; B01J 19/0013; F28D 21/0001; C25B 1/04; C25B 9/65; C25B 15/081; C01B 2203/0233; C01B 2203/0238; C01B 2203/1241; C01B 2203/0833; C01B 2203/085; C01B 2203/0855; C01B 2203/0866; C01B 2203/0883; C01B 2203/84; C01B 2203/0894; B01J 2219/00108; B01J 2219/00099; B01J 2219/00135; B01J 2219/00139; F28D 20/0056

(57) ABSTRACT

A plant uses one or more renewable energy sources to facilitate the processing of a hydrocarbon to produce hydrogen, syngas or other products. One renewable energy source is solar energy, which may be harnessed by (a) directly heating a thermal storage medium by way of a concentrated solar thermal (CST) plant; (b) converting the solar energy using photovoltaic cells to produce electricity and using the electricity to heat the thermal storage medium, (c) a combination of both, or (d) converting the solar energy using photovoltaic cells to produce electricity and using the electricity to heat a reactor by way of resistive or inductive heating. The thermal storage medium, when used, is arranged to store enough thermal energy to enable 24-hours a day processing of the hydrocarbon. Electricity derived from PV cells may be used to enable the production of heat for processing when radiant energy from the sun is insufficient.

### CROSS-REFERENCE TO RELATED APPLICATIONS

This application is a continuation of International Application No. PCT/AU2021/050329, filed Apr. 9, 2021, which claims priority from AU 2020901146, filed Apr. 9, 2020, and from AU 2020903596, filed Oct. 5, 2020, the disclosures of which are incorporated herein by reference in their entireties.

### TECHNICAL FIELD

A renewable energy hydrocarbon processing method and plant are disclosed. The method and plant use one or more renewable energy sources to produce heat to facilitate the processing of the hydrocarbon. The renewable energy sources may include the sun, the wind, and geothermal heat sources. A possible target hydrocarbon for processing is methane. The methane may be derived from, but not limited to: natural gas, coal seam gas or decomposition of organic material for example from household waste. The hydrocarbon processing may produce, amongst other substances: hydrogen, methanol or syngas.

### BACKGROUND

As concerns for the health of the planet grow hydrogen is being increasingly seen as a highly desirable fuel because its combustion by-product is water only. However, hydrogen is not a natural energy source and must be produced from hydrocarbons or water. Known processes for generating hydrogen include steam reforming where water is reacted with methane; dry reforming where methane is reacted with carbon dioxide; and bi-reforming where methane is reacted with carbon dioxide and water. Often the methane is provided as natural gas. Each of these reforming processes is conducted in the presence of a catalyst and needs substantial heat (i.e., energy) input.

The resultant product of the reforming process includes hydrogen and syngas which can be used for the production of a range of chemicals and fuels and other products including but not limited to: methanol and diesel DME.

Where the heat or energy input for reforming is provided directly or indirectly by the combustion of a hydrocarbon there is an accompanying production of carbon dioxide. This offsets to an extent the benefit of producing hydrogen as a fuel.

Embodiments of the disclosed method and plant seek to provide a method and plant for processing hydrocarbons which reduces the overall carbon footprint having regard to the use of the resultant products of the processing.

The above references to the background art do not constitute an admission that the art forms a part of the common general knowledge of a person of ordinary skill in the art.

### SUMMARY

In broad and general terms, the idea or concept behind the disclosed method and plant is to use one or more renewable energy sources to facilitate the processing of a hydrocarbon to produce hydrogen, syngas or other products. One renewable energy source may be solar energy. The solar energy may be harnessed by (a) directly heating a thermal storage medium by way of a concentrated solar thermal (CST) plant; (b) converting the solar energy using photovoltaic cells to produce electricity and using the electricity to heat the thermal storage medium, or (c) a combination of both, or (d) converting the solar energy using photovoltaic cells to produce electricity and using the electricity to heat a reactor or the reactants by way of resistive or inductive heating. The thermal storage medium, when used, is arranged to store enough thermal energy to enable 24-hours a day processing of the hydrocarbon. Optionally, electricity derived from PV cells, or from other renewable energy powered generators or converters may be used, directly, or via a battery, to provide or enable the production of heat to continue the processing when, for example due to inclement weather for an extended period, radiant energy from the sun by itself would otherwise be insufficient to do so, thus also provide 24 hours per day processing.

The method and system adopt energy integration strategies to maximise the energy efficiency. This may involve for example using waste heat from one process or process stream as a thermal energy source for another process or process stream either upstream or downstream of the first mention process or process stream. In another example energy captured from the solar radiation by way of a CST plant or the PV cells may be used in aspects of the method and system to generate reactants for the hydrocarbon processing. For example, in the case of processing methane the solar radiation can be used to power an electrolysis system to produce oxygen that may be used as a reactant for an auto thermal reactor, or to facilitate a cleaner burning of methane to provide a supplemental heat source.

The hydrocarbon processing includes but is not limited to reforming of hydrocarbon, such as, but not exclusively, methane. The reforming may be: steam reforming, dry reforming, or bi-reforming. This may be performed in more than one step or reactor. Heat energy for these endothermic reforming processes may be provided by, but is not limited to, one or a combination of two or more of the following mechanisms:

(a) direct heat transfer from the thermal storage medium by conduction to the reactor in which the reforming process occurs;

(b) transfer of heat from the thermal storage medium to a thermal transfer fluid which subsequently transfers heat by conduction to the reactor;

(c) electrical resistance heating of the reactor and thus reactants flowing through the reactor;

(d) electrical inductive heating of the reactor utilising for example, but not limited to, magnetic particles or electrically conductive particles embedded in a catalyst in the reactor, and electrical current conducting coils located within or about the reactor;

(e) electrical resistance heating or electrical inductive heating of a heat storage medium which subsequently transfers heat by conduction either directly or via a separate heat transfer fluid, to the reactants.

As a contingency in the event of a prolonged period with low solar radiant energy availability, embodiments of the disclosed method and plant may continue operation by use of supplemental thermal energy derived from the combustion of some of the hydrocarbon which would otherwise be processed. The heat generated from this combustion may be applied directly to the thermal storage medium or to a reformer reactor. In either case if the method and plant utilise a thermal transfer fluid then this fluid may be used to transfer heat from the combustion between the thermal storage medium and the reformer reactor. For example, if the heat from the combustion is applied to the thermal storage fluid then the thermal transfer fluid may be used to transfer heat to the reformer reactor. Alternately if heat from the combustion is applied to the reformer reactor then a thermal transfer fluid which also flows through the reformer reactor may be used to subsequently transfer heat to the thermal storage medium. When the method and plant also includes the generation of oxygen from an electrolysis system, this oxygen may be added to the hydrocarbon before or during the combustion.

The integration and prudent management of heat produced by the one or more renewable energy sources in the disclosed method and plant enhances production efficiency and economics, as well as minimising carbon production.

In a first aspect there is disclosed a method of processing a hydrocarbon comprising: heating a heat storage medium by using thermal energy derived from one or more renewable energy sources; heating one or more reactants including at least a hydrocarbon in at least one reactor by flowing the heat storage medium or a heat transfer medium in thermal contact with the heat storage medium through the at least one reactor to elevate the temperature of the reactants to above a threshold temperature ≥T1 needed to cause a reaction in the at least one reactor to process the hydrocarbon to produce a target product; and pre-heating at least one of the reactants using heat subsequential to the reaction in the at least one reactor.

In a second aspect there is disclosed a method of processing a hydrocarbon to produce methanol or other synthesis chemicals comprising:

heating a flow of a heat transfer medium by using thermal energy derived from one or more renewable energy sources to produce a flow of a superheated heat transfer medium at a threshold temperature≥T1; using the flow of the superheated transfer medium to heat in parallel:

(a) a reactant feed stream including the hydrocarbon to produce a superheated reactant feed stream;

(b) a catalyst and the superheated feed stream when in a reactor to produce syngas; and

(c) a heat storage medium.

In a third aspect there is disclosed a method of processing methane to produce a target product comprising:

harnessing energy from a renewable energy source to produce a superheated heat transfer medium;

when the superheated heat transfer medium is at a threshold temperature ≥T1:

a) super heating a reactant stream which includes the natural gas as using a first stream of the superheated heat transfer medium;

b) heating the superheated reactant stream and a catalyst when in a reactor using a second stream of the superheated heat transfer medium; and

c) transferring heat to a heat storage medium using a third stream of the superheated heat transfer medium; and

when the superheated heat transfer medium is below the threshold temperature T1:

d) using heat from the thermal energy storage medium to produce a feed stream of superheated transfer medium a temperature ≥T1;

e) using the superheated heat transfer medium produced by heat transfer from the heat storage medium to superheat the reactant stream, and heat the superheated reactant stream and the catalyst when in the reactor.

In a fourth aspect there is disclosed a method of processing methane comprising:

heating a flow of a heat transfer medium by using a concentrated solar thermal (CST) heater to produce a flow of a superheated heat transfer medium;

using the flow of the superheated transfer medium to heat in parallel:

(a) a reactant which includes natural gas flowing into a reactor to produce syngas;

(b) feed stream of the reactant prior to flowing into the reactor, to produce a superheated reactant stream; and

transferring heat from a flow of syngas produced in the reactor to preheat the feed stream prior to producing the superheated heat transfer medium.

In a fifth aspect there is disclosed a plant for processing a hydrocarbon comprising:

a heat transfer system powered by one or more renewable energy sources and arranged heat to a heat transfer medium;

a closed circuit configured to circulate the heat transfer medium through the heat transfer system to produce a superheated heat transfer medium stream;

a reactor for reforming a reactant stream which includes the hydrocarbon; and

a heat exchanger;

wherein the closed circuit is further arranged to circulate a parallel flow of the superheated heat transfer medium through the reactor and the heat exchanger.

In a sixth aspect there is disclosed a plant for processing a hydrocarbon comprising:

a closed loop primary energy circuit through which a heat storage medium circulates, the primary energy circuit including an energy conversion system arranged to produce heat from one or more renewable energy sources and transfer that heat to the circulating heat storage medium;

a reformer circuit arranged to conduct one or more reactions for reforming the hydrocarbon to produce hydrogen, the primary energy circuit being arranged to transfer heat to the reformer circuit to promote the one or more reactions; and

an electrolyser powered by energy sourced from the energy conversion system, the electrolysis station producing additional hydrogen to augment the hydrogen produced by the reformer circuit.

In a seventh aspect there is disclosed a method of reforming a hydrocarbon to produce hydrogen comprising:

circulating a heat storage medium through an energy conversion system arranged to produce heat from one or more renewable energy sources and transfer that heat to the circulating heat storage medium;

transferring heat from the heat storage medium to a reformer circuit arranged to reform the hydrocarbon to produce hydrogen; and

using energy derived from the energy conversion system to drive an electrolyser to produce additional hydrogen.

In an eight aspect there is disclosed an inductively heated reactor for facilitating a chemical reaction comprising a one or more flow paths through which reactants for the chemical reaction flow, a volume of electrically conductive or ferromagnetic material surrounding the flow paths, and an electric coil wound around the flow paths.

In one embodiment the volume of electrically conductive or ferromagnetic material comprises a solid block of electrically conductive or ferromagnetic material and the flow paths comprise passages extending through the solid block.

In one embodiment the inductively heated reactor may comprise an outer body through which the one or more flow paths extend, and the volume of electrically conductive or ferromagnetic material is in the form of a particulate or powder material disposed within the outer body and surrounding the flow paths.

In one embodiment the one or more flow paths comprise pipes or conduits made of a thermally conductive material.

In one embodiment the one or more flow paths contain a catalyst for facilitating the chemical reaction.

In a ninth aspect there is disclosed an inductively heated reactor comprising a body containing one or more flow paths through which reactants for a chemical reaction can flow, at least one of the flow paths flow lined with or containing a catalyst which the reactants contact when flowing through the flow path, and wherein the catalyst includes a plurality of embedded or mixed electrically conductive or ferromagnetic material; and a coil for conducting an electric current surrounding the flow paths.

### BRIEF DESCRIPTION OF THE DRAWINGS

Notwithstanding any other forms which may fall within the scope of the method and plant as set forth in the Summary, specific embodiments will now be described, by way of example only, with reference to becoming drawings in which:

FIG. 1 is a process flow diagram illustrating in a broad and general sense aspects of the disclosed method and plant for solar hydrocarbon processing;

FIG. 2 is a schematic representation of a first embodiment of the disclosed method and system when operated during daylight hours;

FIG. 3 is a schematic representation of the first embodiment shown in FIG. 2 but when operated in a night mode;

FIG. 4 is a schematic representation of a second embodiment of the disclosed method and system when operated during daylight hours;

FIG. 5 is a schematic representation of the first embodiment shown in FIG. 4 but when operated in a night mode;

FIG. 6 shows a possible variation of the system and depicted in FIG. 2;

FIGS. 7a and 7b: are a schematic representation of a third embodiment of the disclosed method and system having a flow of a single thermal medium which is directly heated by the renewable energy source acts as both a heat transfer medium used to provide heat to facilitate a chemical transformation of the reactant feed and a thermal energy storage medium to enable continuation of the chemical transformation notwithstanding a diurnal nature of the renewable energy source;

FIGS. 8a and 8b are a schematic representation of a fourth embodiment of the disclosed method and system utilising a combination of a pre-reformer to affect partial reforming and heating of a feed stream prior to full reforming;

FIG. 9 is a schematic representation of a further embodiment of embodiment of the disclosed method and plant arranged process methane by way of reforming using a CST plant and incorporating an auto thermal reactor and electrolysis system;

FIG. 10a is a schematic representation of one form of inductively heated reactor that may be used in embodiments of the disclosed plant and method; and

FIG. 10b is a schematic representation of a second you form of inductively heated reactor that may be used in embodiments of the disclosed plants method.

### DETAILED DESCRIPTION

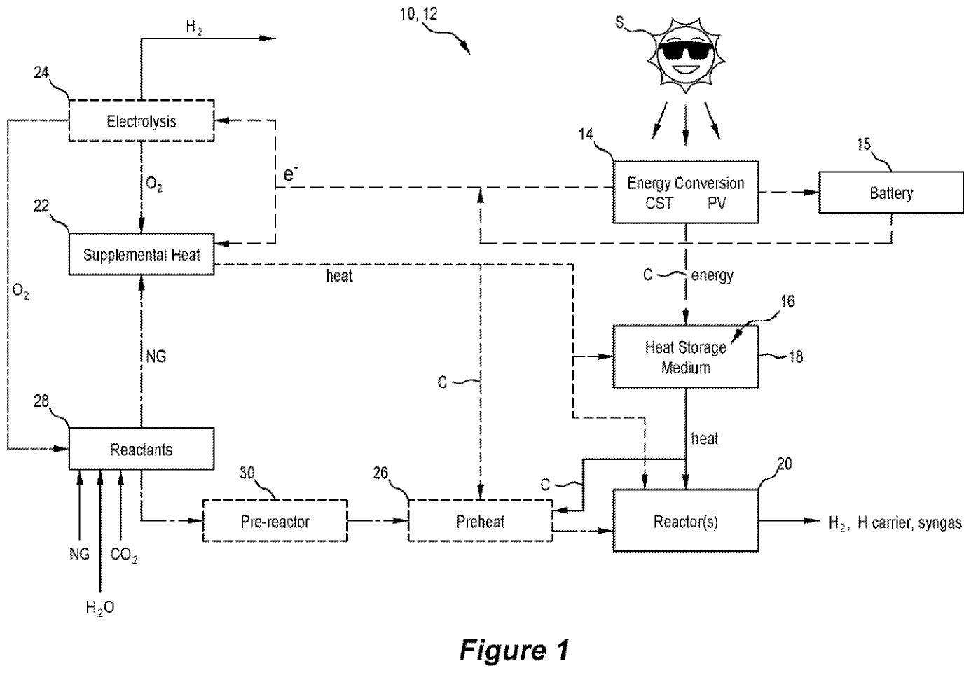

FIG. 1 is a process diagram illustrating in a general sense, aspects of the disclosed method 10 and associated plant 12 for hydrocarbon processing using renewable energy as a primary energy or heat source. In one or more embodiments the hydrocarbon may be methane. The source of the methane is not critical to aspects of the disclosed method and plant. Possible sources include natural gas recovered from subterranean reservoirs, coal seam gas and methane harvested from decomposition of organic materials such as household garbage.

In this embodiment the primary renewable energy source is solar energy from the sun S. The solar energy is converted at a renewable energy conversion system 14 into one or both of: heat using a concentrated solar thermal (CST) plant; and, electricity using for example photovoltaic cells. When both CST and photovoltaic cells are part of the energy conversion system 14, at least some of the electricity generated by photovoltaic cells can be passed to and stored in a battery 15.

In other embodiments the renewable energy source may be, or may include, a geothermal heat source, wind turbine generators, or ocean wave powered generators. So, in some embodiments the energy conversion system 14 may include one or a combination of any two of more of:

a CST plant

PV cell

Geothermal heat source

Wind turbine generator

Ocean wave generator

For ease of description the present embodiments the energy conversion system 14 is described as a combination of a CST plant and PV cells.

An energy or heat transfer circuit C facilitates the transfer of heat/thermal energy from the energy conversion system 14 throughout the plant 12 to enable the processing of the hydrocarbon. Energy derived from the energy conversion system 14 is available and/or stored to drive or power the plant 12 on a 24 hours per day basis through the heat transfer circuit C. In this specific embodiment where a CST plant is included in the energy conversion system 14 is the heat transfer circuit C includes a volume of a heat storage medium (HSM) 16. At any one time a substantive volume of the HSM 16 is held in a heat storage facility 18. Heat from the storage facility 18 is available to drive or power the plant 12 on a 24 hours per day basis through the heat transfer circuit C.

The transfer of heat by the heat transfer circuit C to the heat storage facility 18 and the rest of the plant 12 can be performed in a number of different ways as explained below.

1. Single heat storage medium/heat transfer fluid. In one embodiment of the method 10 and plant 12, the heat storage medium 16 also zo acts a heat transfer fluid. That is, the heat storage medium 16 is the same medium as the heat transfer fluid and circulates through the circuit C to transfer heat to parts of the plant beyond the storage facility 18 and energy conversion system 14.

2. Different heat storage medium and heat transfer fluid. In other embodiments heat may be transferred through the circuit C using a heat transfer fluid that is physically separate from heat storage medium. While the HSM 16 and the heat transfer fluid can be the same in terms of their composition and material type, it is envisaged that in most instances they will be different in one or both of composition and material type. For example, the HSM 16 may be in the form of a molten salt, or a particulate solid material while the heat transfer fluid may be water, steam or a gas or gas mixture.

In such embodiments the heat transfer fluid and the heat storage medium 16 may flow in different sub-circuits of the heat transfer circuit C which are thermally coupled, for example by one or more common heat exchangers to transfer heat between the heat transfer fluid and the heat storage medium 16.

This may be termed as an “indirect” heat transfer method/mechanism because the heat for the purposes of facilitating a chemical reaction for processing of a hydrocarbon is derived from a heat transfer fluid which itself is heated by the thermal storage medium, rather than being heated directly by the energy conversion system 14.

In option 1, the heat storage medium 16 is heated directly by energy transfer from the energy conversion system 14. This may be for example by circulating the HSM 16 via the circuit C through a heat receiver of a CST plant; and/or by heat derived through electricity produced by the PV cells or the battery 15. In option 2, the heat storage medium 16 is heated by heat transfer from the separate heat transfer fluid that circulates through the energy conversion system 14 CST plant, or is heated by electricity from the PV cells/battery 15. It is also possible in option 2 to heat the HSM 16 directly by action of the energy conversion system. In this instance the HSM 16 is heated by both the direct action of the energy conversion system 14 and via the heat transfer fluid.

The heat transfer fluid and the heat storage medium may be in the form of a particulate solid material, a molten salt or a superheated liquid or vapour or gas or mixed liquid and vapour phase fluid.

When the heat transfer fluid and the heat storage medium are one and the same (as per option 1 above) that may circulate in the common heat transfer circuit C. In embodiments where the heat transfer fluid and the heat storage medium circulate in physically separate but thermally coupled sub-circuits (as per option 2, above) the transfer fluid and the storage medium can be selected for optimum performance having regard to their different functions.

For the purposes of storing heat in the heat storage facility 18 the heat storage medium 16 may be in the form of a particulate solid which has greater heat capacity than say a liquid or gas at the same pressure. On the other hand, for the purposes of transferring heat from the heat storage medium 16 to other equipment or components of the plant 12 the heat transfer fluid may be in the form of a liquid or gas which can dissipate heat much more quickly than a solid. The idea here is to use the heat storage medium 16 as a heat source (i.e., energy supply) for the plant 12 when solar energy is not available.

The plant 12 includes at least one reactor 20 in which the hydrocarbon processing occurs. Heat transfer to the reactor 20 can be by direct thermal conduction from the heat storage medium 16 via the circuit C (i.e., under option 1, above). This would involve for example the heat transfer medium 16 flowing through the reactor 20 which would heat the reactor, and consequently catalyst and reactants 28 in the reactor. Alternately the heat transfer can be indirect using a heat transfer fluid which is heated by the heat storage medium 16 and then transfers heat through a sub circuit to the reactor 20 (i.e., option 2, above). The thermal transfer fluid may be in the form of for example superheated steam or carbon dioxide.

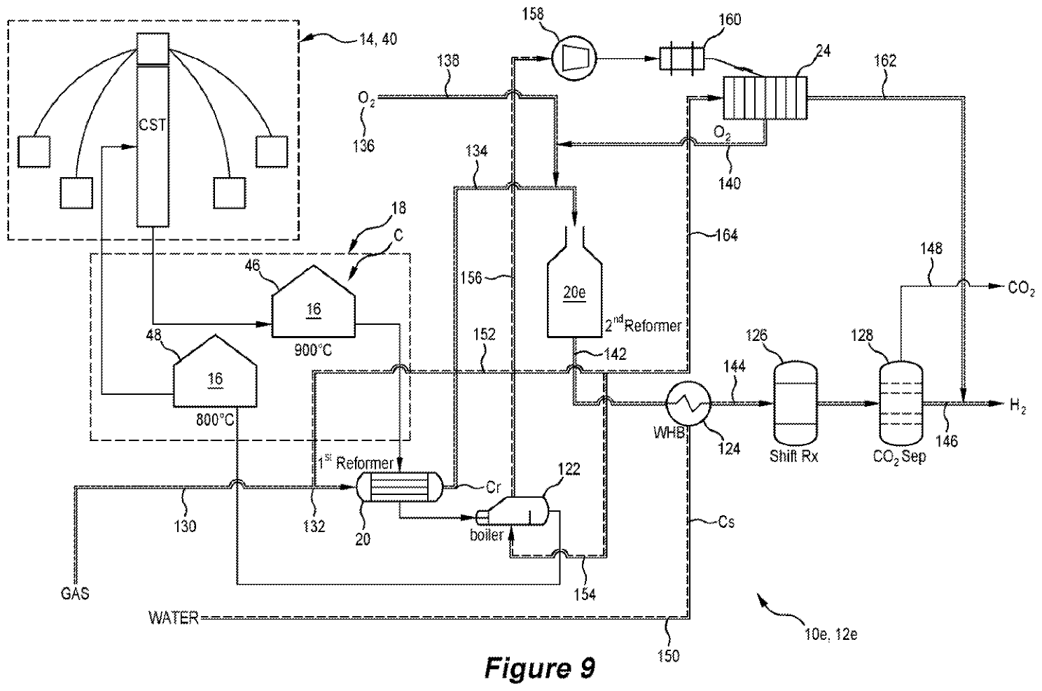

The indirect heat transfer mechanism (i.e., option 2) may be well suited to when the plant 12 is designed to have a relatively high heat demand or capacity such that the heat storage medium 16 needs to be capable of holding relatively high temperatures such as for example over 900° C., or over 1000° C. or higher such as over 1200° C. To withstand such temperatures the flow path and equipment (for example pipes or other mass transfer devices and valves) may need to be custom engineered which adds cost and complexity to the plant 12. This can be minimised when the only section of the circuit C than needs withstand such temperatures is that running between the energy conversion system 14 and the heat storage facility 18. An example of this is described later with reference to FIG. 9. A benefit of using a heat storage medium 16 that can hold such relatively high temperatures is that it has a greater thermally storage capacity per unit mass or volume of the medium. This may allow the method and plant 12 to operate during extended periods when solar radiation is below optimum.

Embodiment of the method 10 and plant 12 also contemplate the provision of a supplemental heat source 22 to be made available to enable continued hydrocarbon processing. The heat from the supplement heat source 22 may be made available to one of or both the reactor 20 and the heat storage medium 16. The supplement heat can be generated by resistive or inductive heaters in the reactor 20 or the heat storage facility 18. The heaters may be powered by electricity from the battery 15. Additionally, or alternately the supplement heat from source 22 can be generated by the combustion of some of the hydrocarbon which would otherwise be processed. For example, some of the hydrocarbon reactant can be combusted to produce heat for heating the reactor 20 or the heat storage medium 16 in the facility 18. Also, oxygen may be sourced from an optional electrolyser 24 and mixed with the hydrocarbon to enhance the combustion process and/or assist in reducing the production of carbon-based gases. The supplemental heat can be used when there is insufficient solar energy falling on the plant 12 to heat the heat storage medium 16 by way of the CST plant alone.

Either one or both the heat storage medium 16 and the supplemental heat source 22 can also be used to provide thermal energy to an optional pre-heater 26. The pre-heater 26 may be used for example to heat the reactants prior to them reaching the reactor 20. This reduces the temperature differential between the reactants 28 and the heat storage medium 16/reactor 20 thereby reducing the thermal load on the heat storage medium 16. This in turn enables the heat storage medium 16 to hold heat at higher temperatures for a longer period.

The reactants 28 supplied to the reactor 20 may include in addition to the hydrocarbon (in this example methane from natural gas) other reactants needed to produce the desired target products. In this example the reactants may include water, carbon dioxide, and oxygen. In one embodiment the oxygen may be provided to an auto thermal reforming reactor which may be used in an embodiment in addition to the reactor 20 (for example as shown in the embodiment illustrated in FIG. 9).

In other variations described later, embodiments of the method 10 and plant 12 may include an optional pre-reactor 30 to partially conduct the reforming process with the rest of the reforming process being completed in the reactor 20. In this variation heat to drive the pre-reactor may be sourced from syngas produced by the reactor 20. In this variation the end product of the processing may be methanol.

Examples of more specific embodiments of the method 10 and plant 12 which incorporate various combinations of the aspects described above will now be described.

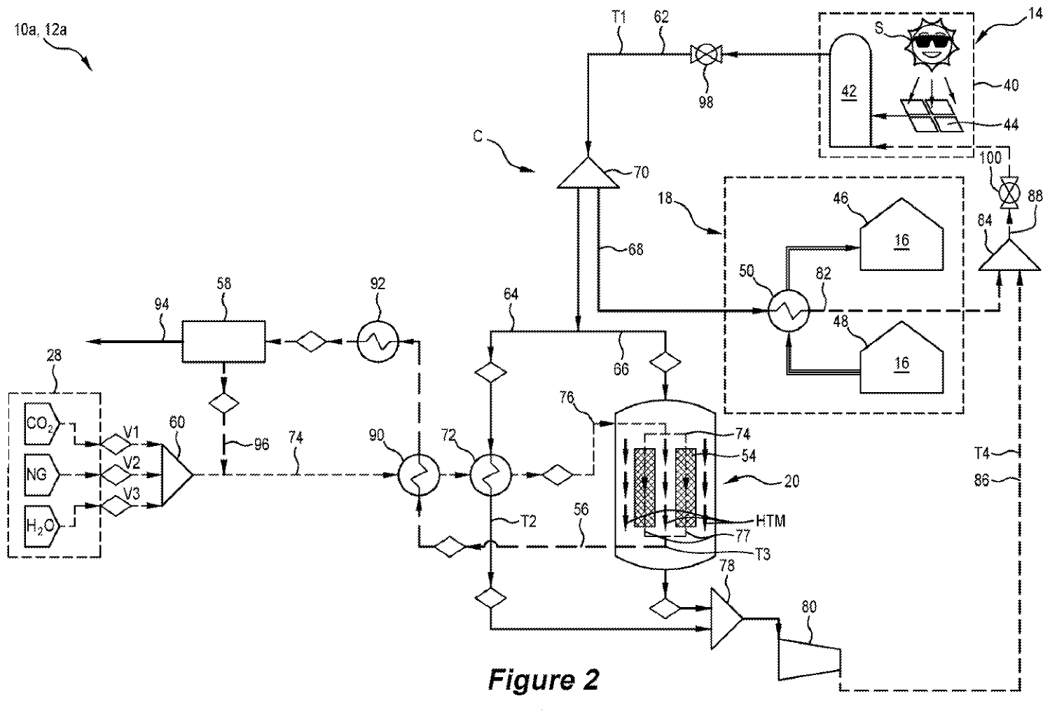

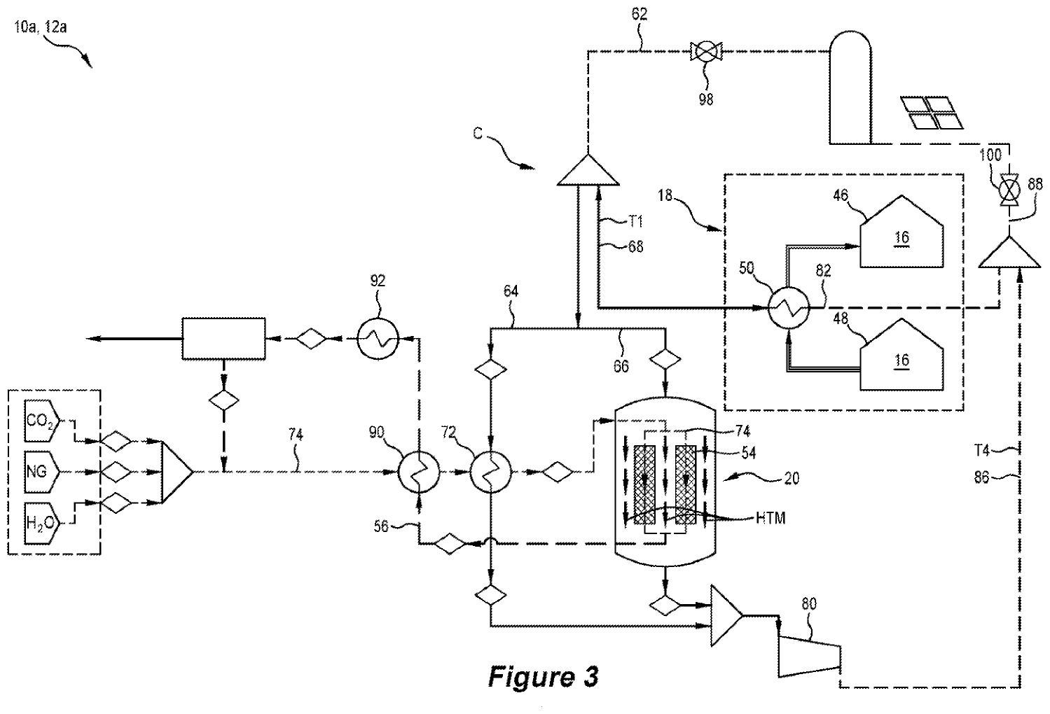

FIGS. 2 and 3 schematically illustrate one embodiment of the disclosed method 10a and corresponding plant 12a for solar processing of a hydrocarbon, in the form of methane sourced for natural gas. In this embodiment the processing is the bi-reforming of methane to produce methanol. FIGS. 2 and 3 shows the heat and process flow during daylight hours and night time hours, respectively.

The energy conversion system 14 is in the form of a CST plant 40 which includes a solar receiver 42 and a movable bank of mirrors or heliostat 44. The heat storage facility 18 comprises: a hot tank 46 and a cold tank 48, which between them hold a volume of the HSM 16; and, a heat exchanger 50 through which the HSM 16 can flow between the tanks 46 and 48.

The circuit C circulates a heat transfer medium/fluid (HTM) which is physically separate from (i.e., does not physically mix with) but is thermally coupled to the HSM through the heat exchanger 50. In all embodiments the HTM and the HSM may include but are not limited to: superheated steam; other mixed gas/vapour/liquid phase fluid; a single phase liquid; a molten salt such as calcium nitrate, potassium nitrate or sodium nitrate; or solid particulate material such as, but not limited to bauxite. However, in this embodiment the HTM and HSM are different from each other, with the HTM being a superheated steam and the HSM being either a molten salt or a solid particulate material.

The plant 12a includes a reactor in the form of a reformer 20 which receives reactants 28 conducted through a feed stream path 74, 76. A catalyst 54 is held within the reformer 20. More particularly the catalyst 54 is contained within conduits 77 forming the paths 74. In one example of a catalyst 54 lines an inside surface of the conduits 77. When the reactants 28 contact the catalyst 54 at a sufficiently high temperature the reactants are chemically transformed into syngas being a gas mixture comprising primarily of hydrogen, carbon monoxide and unreacted reactant. The syngas leaves the reformer 20 and flows through a flow path 56 to a synthesis reactor module 58. In this example the reactor module 58 is one capable of producing methanol. It should be appreciated however that the module 58 can be varied depending on the desired end product. For example it may be desired to produce another product such as ammonia or other hydrogen carrier.

In this embodiment the reactants 28 comprise a combination of: carbon dioxide (CO₂); natural gas (NG), as the methane source; and, water (H₂O), all being fed to a mixer 60. The relative volumes of the reactants fed to the mixer 60 may be varied by control of respective valves V1, V2 and V3. Also, the reactants may be provided at different temperatures to the mixer 60. Although not shown in the present drawings further heat integration and utilisation may be achieved by the transfer of heat to or from the individual reactants and the process fluids (the syngas and methanol).

A closed heat transfer circuit C transfers thermal energy from the energy conversion system 14 to the thermal storage facility 18 which holds the heat storage medium (HSM) 16, and to the reminder of the plant 12a. The circuit C divides the HTM which flows from the energy conversion system 14 at temperature T1 for example 1200° C. through a path/conduit 62, into three streams: 64, 66 and 68. A flow splitter 70 splits the flow path 62 into a first portion that includes the stream 68, and a second portion which subsequently splits into the streams 64 and 66.

The stream 64 passes through a heat exchanger 72 and transfers heat from the HTM to the reactants 28 flowing as reactant feed stream 74. This produces a superheated reactant feed stream that flows through path 76 into the reactor 20. Due to the transfer of heat the HTM leaves the heat exchanger 72 at a lower temperature T2, (for example, but not limited to, about 750° C.) and flows to a flow combiner 78 of the circuit C.

The stream 66 carrying HTM at temperature T1 flows through the reactor 20 to heat the reactants 28 and the catalyst 42. The HTM in stream 66 leaves the reactor 20 at a temperature T3 and flow to the flow combiner 78. The temperature T3 may for example be in the order of 950° C.

The combined HTM streams 64 and 66 drive a generator 80. The generator 80 is used to generate electrical power. The electrical power may be used in a variety of ways including but not limited to: powering utilities or other equipment at the plant 12a; feeding to an AC grid, through an inverter if necessary; stored in a battery to provide a stored power source that may be used for a variety of functions including to heat the HTM; power an electrolyser; or any combination thereof.

The HTM in the combined streams 64, 66 after exiting the generator 80 is returned via a cold path 86 of the circuit C to flow back to the energy conversion system 14. The HTM in the path 86 is at a temperature T4, which may for example be in the order of about 725° C. or lower depending on the renewable energy converter design.

The HTM in the path 68 flows to a heat exchanger 50 and transfers heat to the HSM 16 in the storage facility 18. In particular, the HSM flows from the cold storage tank 48 to the warm storage tank 46 via the heat exchanger 50 where it is heated by the HTM. The HTM in stream 68 then flows through a path 82 of the circuit C and mixes in a combiner 84 with HTM flowing through path 86. The combined HTM stream 88 flows through the solar receiver 42 where it is reheated back to the temperature T1. The HTM continuously cycles through the circuit C and thus is cyclically heated to a temperature T1 and then cooled by the transferring heat to the:

reactant 28 and catalyst 42 in the reactor 20,

reactant 28 via the heat exchanger 72 to produce a preheated reactant feed stream, and

heat storage medium in the heat storage facility 18.

The plant 12a also includes a heat exchanger 90 designed to transfer heat from the syngas at temperature T3 flowing through the path 56 to the reactant feed stream 74 at a location upstream of the heat exchanger 72. This results in cooling of the syngas, and a pre-heating of the reactant feed stream 74. Thus, the reactant feed stream 74 is heated in two stages prior to entering the reactor 20, these being: (a) by the syngas in the exchanger 90, and (b) by the HTM in heat exchanger 72.

The syngas after passing through heat exchanger 90 flows to a heat exchanger 92 where it is further cooled to a temperature of about 40° prior to entering the methanol synthesis module 58 which produces a methanol stream 94. Any unreacted syngas in the module 58 is transferred via flow path 96 back to the reactant feed stream 74 and thereby carried to the reformer 20.

The heat transfer circuit C includes a valve 98 in the path 62, and a valve 100 in the path 88. When the sun S is shining or otherwise providing solar energy to the energy conversion system 14 the valves 98 and 100 are locked open. This enables a continuous flow of the HTF through the solar receiver to be heated to the temperature T1.

However, when there is insufficient sunlight to heat the HTM to the threshold temperature T1, the plant 12a switches to a “night” mode. This involves the valves 98 and 100 being locked closed altering the flow path in heat transfer circuit C, as shown in FIG. 3. Now the thermal energy held within the heat storage facility 18 is used to heat the HTM 16 to the temperature T1. The night mode may not necessarily coincide with non-daylight hours. Rather method 10a and plant 12a switch to the night mode when the energy produced by the energy converter 14 is insufficient to heat the HTM to a temperature T1. So, this may coincide for example with daylight hours where there is heavy cloud cover including during periods of precipitation.

In the night mode, the heat storage medium flows in a reverse direction to that described above in relation to FIG. 2. That is the flow is from the hot tank 46 through the heat exchanger 50 to the tank 48. Also, because the valves 98 and 100 are now closed the flow direction of the HTM in the paths 82 and 68 is reversed in comparison to those during the daytime. Consequently, the HTM at the lower temperature T4 is now heated by heat transfer from the heat storage medium 16 via the heat exchanger 50. In all other respects the flow of the heat transfer fluid and the flow of the process fluids (i.e., the reactant 28 and the syngas) stays the same as during the daytime.

There may be a reduction in the maximum temperature T1 of the HTM in the night mode because of the inability to add further energy to the system (for example if there is no battery 15, or supplemental heat source 22 as per the plant 12 in FIG. 1). The temperature T1 in the night mode may drop by between 10° and 100° C. In any event the stored thermal energy in the heat storage medium 16/heat storage facility 18 is designed to be sufficient to enable continued production of the methanol or other target product at substantially the same rate as during the daytime.

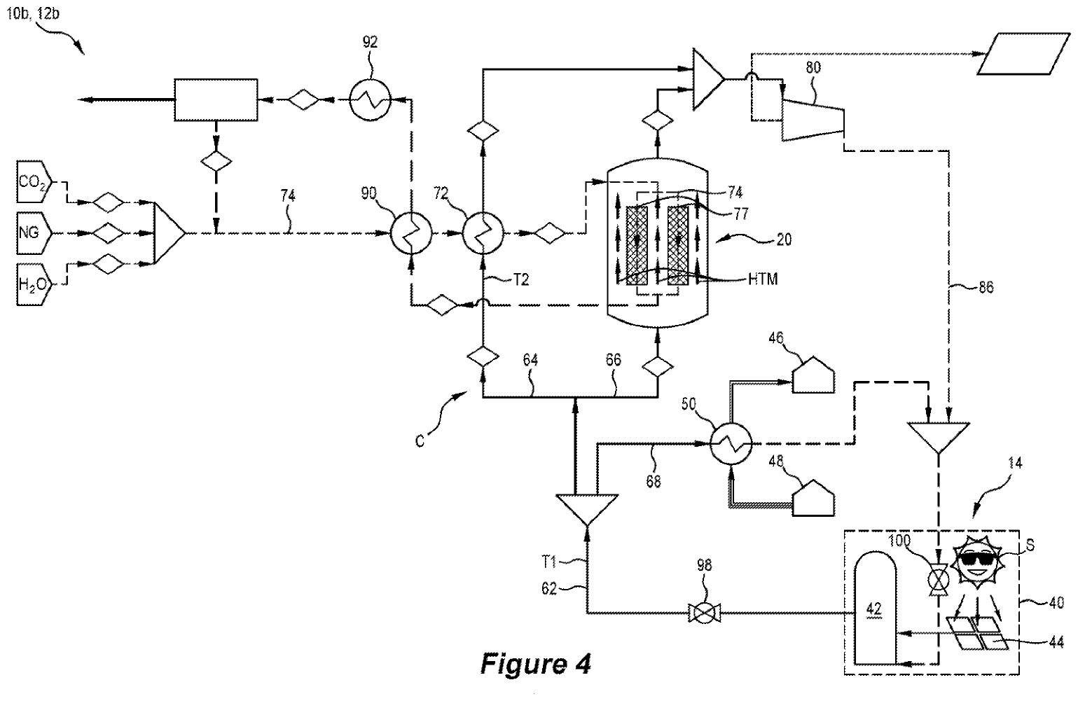

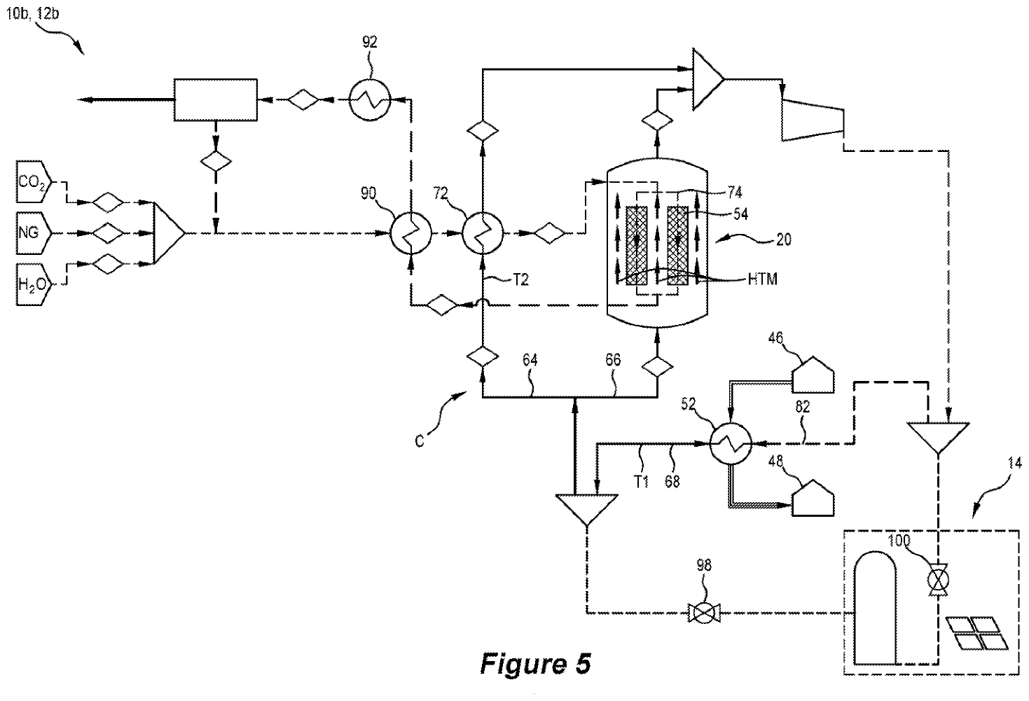

FIGS. 4 and 5 show an alternate embodiment of the method 10b and plant 12b. In these Figures the same reference numbers are used to denote the same features as in the embodiment shown in FIGS. 2 and 3. The only difference between the embodiments is that in method 10b and plant 12b there is a counter current flow of the reactants 28 flow through the conduits 77 and the HTM through the reactor 20. This is to be contrast with the method 10a and plant 12a in which there is a concurrent flow of the reactant 28 and the HTM through the reactor 20. The counter current flow enables greater heat transfer between the HTM and the reactants 28 and thus is more energy efficiency than the concurrent flow. In all other aspects the operation of the method 10b and system 12b is a same as that of the embodiment of FIGS. 2 and 3.

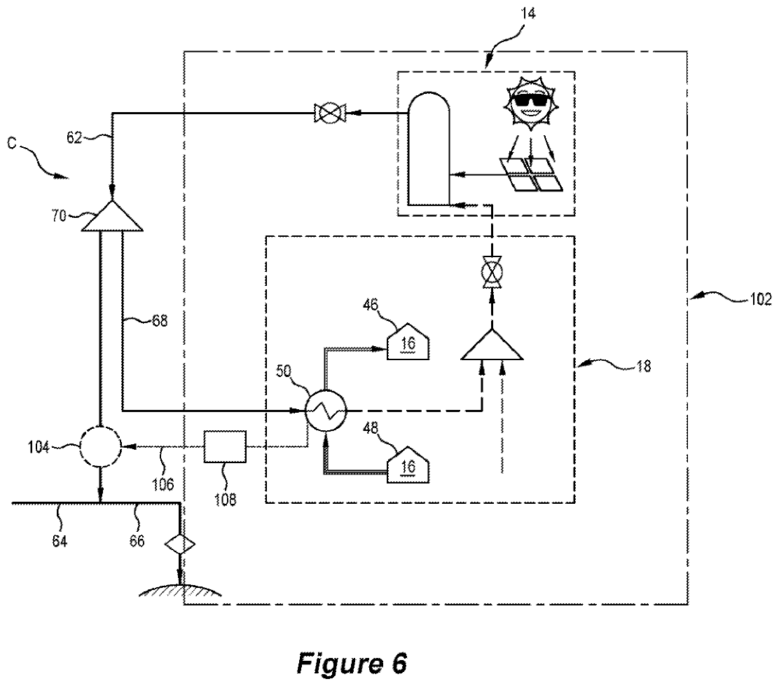

FIG. 6 illustrates a different way of viewing the above described embodiments of the method and system. Here the combination of the heat storage system 18; and the energy conversion system 14 may be considered as forming a thermal energy transfer system 102. When viewed in this manner the plant 12 for producing a product such as but not limited to methanol may be considered as comprising:

(a) the thermal energy transfer system 102 which is powered by a renewable energy source, in this, but not every, embodiment the sun, and arranged to produce thermal energy which is transferred to the heat storage medium 16;

(b) the closed heat transfer circuit C which is configured to circulate the heat transfer medium through the solar energy transfer system 102 to produce a superheated heat transfer medium stream;

(c) the reactor 20 for reforming the reactants 28 which includes methane; and

(d) the heat exchanger 50; and where

(e) the closed circuit C is arranged to circulate a parallel flow of the superheated heat transfer medium through the reactor 20 and the heat exchanger 50.

FIG. 6 also illustrates a possible modification to the previously described embodiments in which the closed circuit C is provided with:

a mixer 104 downstream of the splitter 70 but upstream of both the reactor 20 and heat exchanger 50; and

a flow path 106 from the heat exchanger 50 to the mixer 104, with a valve 108 to selectively open and close the flow path 106.

The general idea behind incorporating the mixer 104, flow path 106 and valve 108 is to enable the thermal energy transfer system 102 to add heat to the heat transfer fluid so prior to splitting into the two streams 64, 66 the heat transfer fluid is at or above the threshold temperature T1. This covers the situation where the energy produced by the energy conversion system 14 for directly heating the heat transfer medium when flowing through the circuit path 62 is not enough for it to reach the threshold temperature T1, but where the temperature is sufficiently close to the threshold such that it can be pushed above the threshold level with heat added from the heat storage facility 18.

A possible use scenario for this is when for example the energy conversion system 14 is a concentrated solar thermal system where during the daytime the radiation provided by the sun is sufficient to only heat the heat transfer fluid to a temperature of example T1-100°, but that the heat transfer fluid could be superheated to the temperature T1 by circulating a part of the flow from the path 62 through the heat exchanger 50 and back to the mixer 104.

This enables the closed circuit C to be selectively controllable to direct the heat transfer fluid to flow:

(a) through the energy conversion system 14 to transfer heat from the sun S to the heat transfer fluid to produce the superheated heat transfer fluid, and subsequently to the HSM 16 in the heat storage facility 18; and where the superheated heat transfer medium can be split into the streams 64 and 66; i.e., no change to heat flow as described above in relation to the embodiments of FIGS. 2 and 4; or

(b) to the heat storage facility 18 to produce the superheated heat storage medium 16, as shown in FIGS. 2 and 4, which when the renewable energy source is of a diurnal nature enables continuation of the process when no or insignificant energy is being converted by the system 14; or

(c) in parallel streams, one through the energy conversion system 14 to transfer heat from the converter 14 to the heat transfer medium to heat the heat transfer medium to a temperature below the temperature T1, and another through the thermal energy storage system 102 to transfer heat from the heat storage medium 16/facility 18 to the heat transfer medium to heat the heat transfer medium to a temperature above the temperature T1; and combining the streams to produce the superheated heat transfer medium stream, (as shown for example in FIG. 6 via the inclusion of the mixer 104, flow path 106 and valve 108).

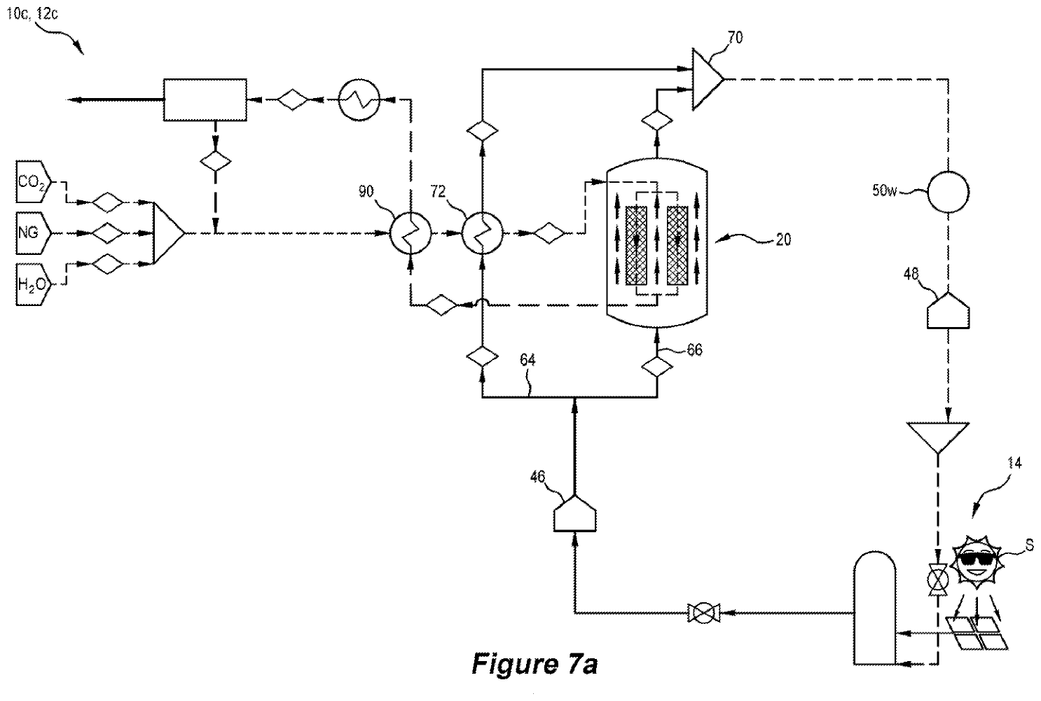

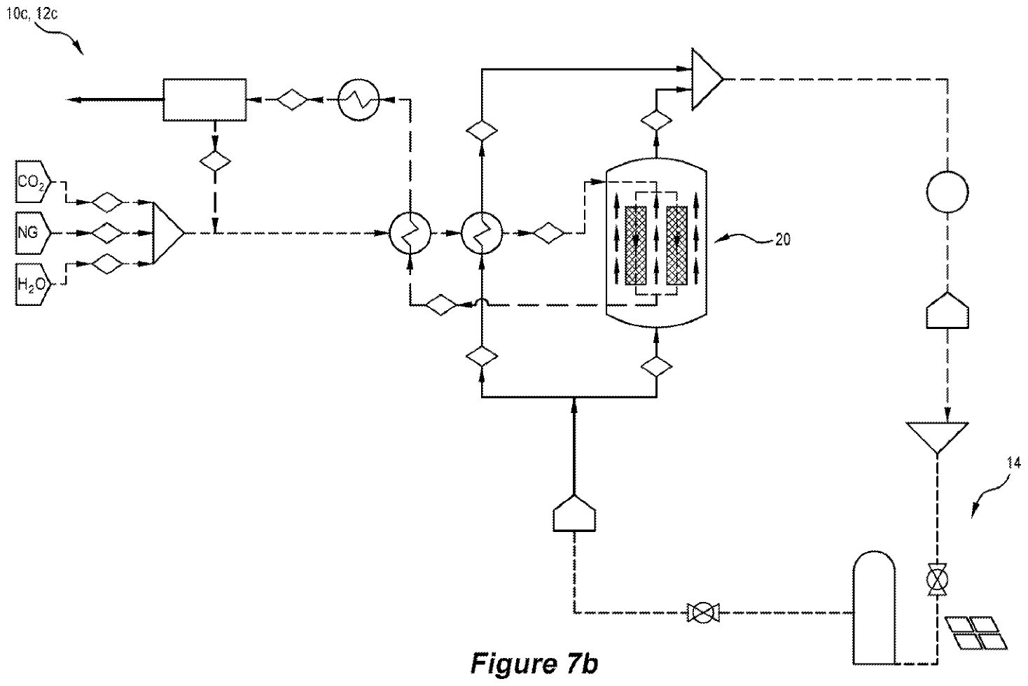

FIGS. 7a and 7b depict a further embodiment of the disclosed method and system 10c, 12c in which the same reference numbers are used to denote the same features as in the embodiment shown in FIGS. 1-5. The only differences between the embodiments is that in method 10c and plant 12c there is a single common heat transfer fluid/medium and heat storage medium (i.e. the medium is one and the same for heat transfer and heat storage), the heat exchanger 50 is replaced by an optional waste heat boiler 50w, and the generator 80 is omitted. The single heat transfer/storage medium may be termed as a direct thermal medium (DTM). The DTM is the only medium that circulates through the circuit C. The DTM is heated by the sun S and/or electricity from PV cells via the energy conversion system 14. The combined volume of the DTM held at any one instant in storage tanks 46 and 48 may be arranged to be greater than a volume of the DTM that is circulated in 24 hours through the plant 12 plus the instantaneous volume of the DTM circulating in circuit C and equipment of the plant 12 other than the tanks 46 and 48 themselves. In this way the tanks and in particular the warm tank 46 hold sufficient heat to continue operation of the plant 12 for an extended period of time, for example up to a week without additional heat input from the system 14 during that period.

Other renewable energy conversion devices and systems may be incorporated in the energy conversion system 14 to provide additional energy sources to heat the DTM. For example one or more wind turbines may be operable to generate electricity which is fed to a battery for subsequent use to heat the heat storage medium/DTM in any one or more of the tanks 46, 48, the reactor 20, or indeed at one more locations along the circuit C through which the medium flows. The heating may be by way of one or both of inductive or resistive heating. Different heating mechanisms may be used in different parts of the plant 12c. For example, resistive or inductive heating of the heat transfer medium/fluid in the tanks 46, 48 and/or the reactor 20. Indeed, as explained in greater detail below in an alternate embodiment resistive or inductive heating can be used as the sole heat/energy transfer mechanism to the reactants inside and outside (for example for preheating) the reactor 20 thereby doing away with the need for the flow and storage of a heat storage medium and a heat transfer fluid. The use of resistive or inductive heating can greatly reduce the footprint of the reactor and thus the plant 10 and improve heat/energy efficiency. This is described in more detail for example in Wismann et al., Science 364, 756-759 (2019); “Electrified methane reforming: A compact approach to greener industrial hydrogen production”

In the plant 12c, during the day and starting arbitrarily at the warm tank 46, the DTM circulates through the circuit C from the tank 46 and splits into two streams 64 and 66. The stream 66 passes through the reactor 20 to power the reforming or other process, while the stream 64 passes through heat exchanger 72 to preheat the reactants. Both streams combine in the mixer 70 and pass through the heat exchanger 50w and subsequently into the cold tank 48. A different volume of the DTM leaves the bottom of the tank 48 and passes through the energy conversion system 14 where it is reheated to circulate back to the warm tank 46. While the flow rate of DTM into and out of the tank 48 is the same, this flow is for different volumes or stratum of material. The flow is analogous to a bath tub which is filled to a predetermined level and has a tap feeding water into the bath from the top and a drain feeding water out from the bottom at the same rate. An analogous situation occurs in the warm tank 46 in relation to the DTM feeding into the tank and DTM flowing out of the tank.

When the thermal energy in the DTM drops (for example due to the diurnal cycle, as shown in FIG. 7b) the valves 98 and 100 in the circuit C are closed. Now the flow of DTM is from tank 46 to tank 48 through the reactor 20. On the next day, the valves 98 and 100 are opened allowing flow of the DTM through the conversion system 14 and more particularly through the solar receiver.

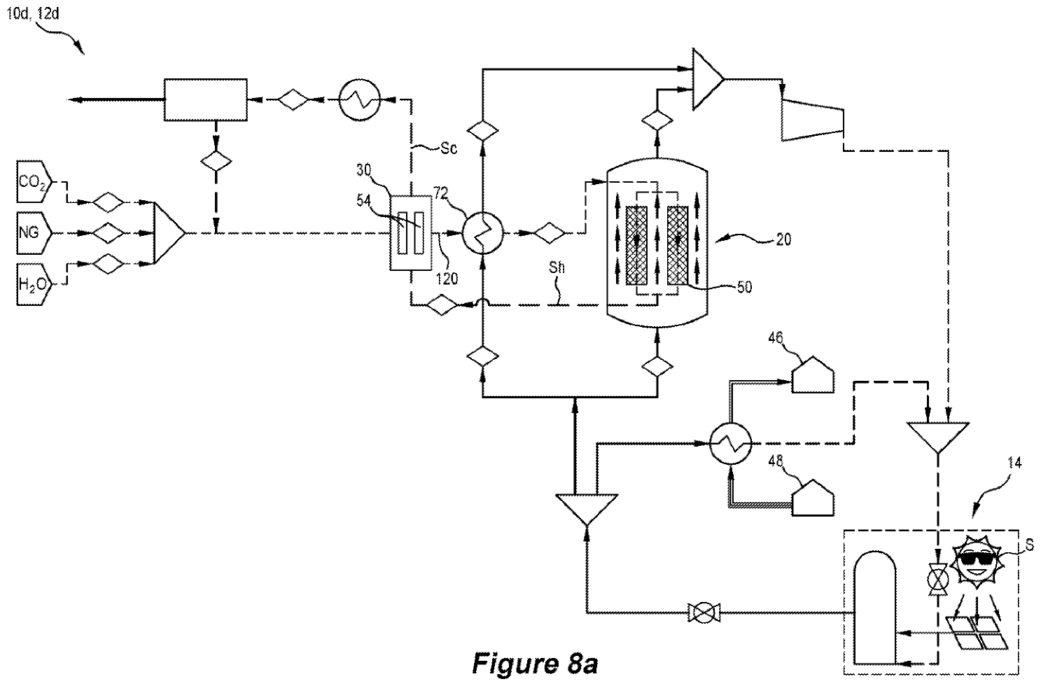

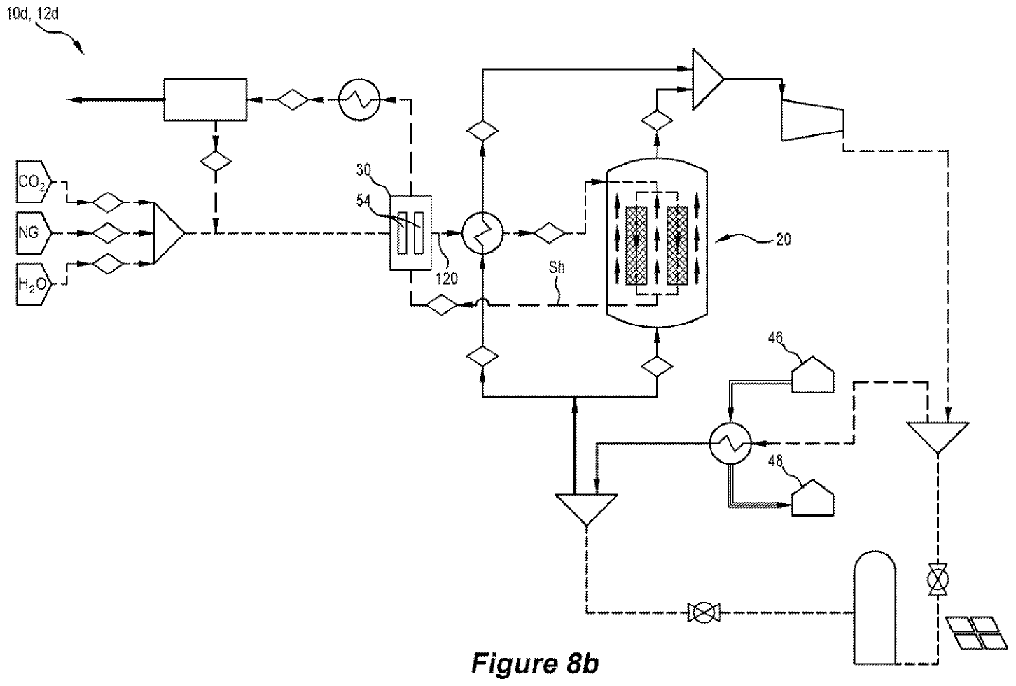

FIGS. 8a and 8b show day and night process flows respectively of yet a further embodiment of the method 10d and plant 12d in which the same reference numbers are used to denote the same features as in the embodiment shown in FIG. 1. The embodiment of FIGS. 8a and 8b differs from that in FIG. 1 by the replacement of the heat exchanger 90 with a gas heated pre-reformer reactor 30. The general idea here is that part of the reforming process will be conducted in the reactor 30 using heat extracted from the syngas from the reformer 20.

The reactor 30 also includes a catalyst 50 for facilitating the chemical reaction of the reactants. A partially reformed reactants stream 120 produced by the reactor 30 is fed to the pre-heater 72 and then the reformer 20. This stream may for example comprise about 30% syngas and 70% unreacted reactant. The 70% unreacted reactant is processed in the reformer 20 to produce a high-grade (optimally 100%) hot syngas stream Sh. This stream Sh after passing through the pre-reformer 30 emerges as a cooled high-grade syngas stream Sc.

FIG. 9 is a schematic representation of another embodiment of the disclosed method 10e and plant 12e. This embodiment uses the direct heat transfer method where the heat transfer medium that is heated by direct action of the energy conversion system 14 flows through the reactor 20 to heat the reactants 28 and the catalyst. Also, this embodiment includes an electrolyser 24 an auto-thermal reactor (ATR) 20e. The electrolyser 24 produces oxygen that is fed to and is consumed in the reaction occurring in, the ATR 10e. Hydrogen produced by the electrolyser 24 is added to the hydrogen output of the plant 12e. The output hydrogen may then be processed or used as desired, for example liquefied, converted to ammonia, converted to methanol etc.

The plant 12e uses a concentrated solar thermal (CST) system 40 in its energy conversion system 14 as the primary heat energy source for powering the plant 12e. The plant 12e has a heat transfer circuit C which forms the flow path for the heat storage medium HSM and includes the energy conversion system 14, the hot storage tank 46, reactor 20, a boiler 122 and the cold storage tank 48. In this circuit C the heat storage medium is circulated through the CST system 40 to absorb heat energy which is then transferred to the reactor 20 to facilitate the processing of the reactants 28, through the boiler 122, the cold storage tank 48 and back to the CST system 40.

In one example the HSM 16 held within the facility 18 is in the form of a molten salt. The HSM 16 after being heated flows into the hot tank 46 at a temperature of about 900° C. The reactor 20 may be in the general form of a shell and tube heat exchanger. In such an embodiment of the HSM flows through the shell side while the reactants 28 flows through the tube side. The HSM leaves the reformer 20 at a lower temperature and then passes through the boiler 122 where it again transfers heat to a heat transfer fluid (typically water/steam), cooling to about 900° C. when it reaches the cold tank 48 prior to being fed again to the CST plant 40.

The storage facility 18, has a capacity substantially greater than the instantaneous volume of thermal fluid circulating through the remaining parts including pipes and conduits of the circuit C. This provides a heat/thermal energy reservoir required for the plant 12e to remain operational 24 hours a day. The instantaneous thermal energy stored in the total volume of the HSM varies with the diurnal cycle but stays sufficiently high to continuously drive the plant 12e.

The plant 12e also includes, a waste heat boiler 124, a shift reactor 126 and a carbon dioxide separator 128. In the first reformer/reactor 20, methane which is provided as a major constituent of a natural gas feed is reacted with water/steam to produce hydrogen and carbon dioxide. The chemical reaction occurring in the reformer 20 is:

CH4+H2O=3H2+CO (reaction 1).

The methane and water in this reaction are the reactants. Natural gas/methane is fed to the reformer 20 via a gas line 130. Water and more particularly steam is injected into the gas line 130 via a valve 132 at a location upstream of the reformer 20. Therefore, a gas/steam mixture is provided to a tube side of the reformer 20 (in this example, the reformer 20 being in the form of a shell and tube heat exchanger). This mixture flows through tubes within the reformer 20 which also hold a catalyst (for example nickel based) to facilitate reaction 1, above. The fluid flowing through tubes is physically isolated from the HSM 16 flowing through the shell side of the reactor 20. The steam at the valve 132 also acts to pre-heat the reactants (i.e., the gas and steam mixture) prior to entry to the reactor 20. The heat in the steam at the valve 132 is derived subsequential to the heat transfer to the reactants in the reactor 20 by the HSM 16. That is, but for the heat transfer occurring in the reactor 20 between the HSM 16 and the reactants which produces the process flow in conduit 134 and subsequently the process flow in conduit 142, the water flowing through the heat exchanger 124 would not be heated. The reactor 20 may optionally be provided with an electric temperature control system which includes an electric heater for keeping the temperature of the reactants in the reactor 20 within a specified range. This control system may run automatically when the thermal energy provided by the thermal fluid is not enough to support the reaction 1 in reformer 20 at a particular rate. The electrical power to drive the electric temperature control system and heater can be provided by a battery (not shown) which is charged by a renewable energy source which forms part of the energy conversion system 14. This will be explained in greater detail later.

The product of the reaction 1, together with any unreacted natural gas/methane flows through a conduit 134 to the second reactor 20e. Oxygen is also separately added to the second reactor 20e. This oxygen may come from one or both of two sources. One source is an oxygen supply (e.g., tanks) 136 which flows through a conduit 138 to the reactor 20e. The electrolyser 24 also produces oxygen and feeds this through a conduit 140 to the reactor 20e. Thus, the reactants in the reactor 20e comprise natural gas/methane, oxygen, water/steam and carbon monoxide. The product of the reaction in the reactor 20e is a syngas comprising a combination of hydrogen and carbon monoxide.

The stoichiometry of the reaction in the reactor 20e is:

CH4+O2+2H2O=10H2+4CO. (Reaction 2)

Reaction 2 is an exothermic reaction generating heat arising from the partial oxidation of the methane and combustion of the oxygen. The outlet temperature of the syngas may be between 950° C. and 1100° C.

The syngas flows through a conduit 142 to and through the waste heat boiler 124. Heat from the syngas is transferred in the boiler 124 to a physically isolated stream of water also flowing through the boiler 124. The transfer of heat converts the water from a liquid phase to the vapour phase (i.e., steam). The flow and function of the steam is described later in the specification.

After passing through the waste heat boiler 124 the syngas flows through a conduit 144 to the shift reactor 126 which converts the carbon monoxide and water in the syngas to carbon dioxide and hydrogen. The stoichiometry of the reaction occurring in the shift reactor 126 is:

CO+H2O=CO2+H2. (Reaction 3).

The carbon dioxide and hydrogen are then separated from each other in the separator 128. The separator 128 has a hydrogen conduit 146 and a carbon dioxide conduit 148 to conduct respective flows of hydrogen and carbon dioxide. The hydrogen from the conduit 146 may be processed as desired, for example liquefied, or converted to hydrogen carriers such as ammonia, methyl cyclohexane or liquid organic hydrogen carrier. The carbon dioxide can be used for example to produce protein for fish meal or may be stored via a sequestration process.

Water is supplied to the waste heat boiler 124 through a water conduit 150. Heat is transferred from the syngas produced by in the reactor 20e to the water to produce a flow of heated water and/or steam. This flow is split into three streams. A first stream flows through a conduit 152 to the valve 132 to produce the gas/steam mixture fed to the first reformer 20.

A second stream flows through a conduit 154 to the boiler 122 where it is heated by the HSM 16 flowing through the circuit C to produce a superheated steam. The superheated steam then flows through a conduit 156 to a turbine 158 that drives a generator 160 producing electricity to power the electrolyser 24. The electrolyser 24 produces the oxygen conducted by the conduit 140 and the hydrogen which flows via a conduit 162 to be added to the hydrogen flowing from the conduit 146. The generator 160 may also feed a battery (not shown) to power other equipment in the plant 12e such as the reformer 20 to facilitate the electric temperature control.

A third stream from the waste heat boiler 124 is fed via a conduit 164 to electrolyser 24. The stream is at an elevated temperate and pressure which improves the efficiency of the electrolysis and pressurizes the oxygen and hydrogen flowing through conduits 140 and 162, respectively.

The combination of the waste heat boiler 124, water boiler 122, and conduits 150, 152, 154, and 164 may be considered as forming a water/steam circuit Cs. (traced in blue phantom line).

The combination of the first reformer 20 and the second reformer 20e, can be considered as forming a reactor circuit Cr (traced in green phantom line). The reactor circuit Cr is the circuit in which methane/natural gas is provided with steam as starting reactants, and hydrogen is produced as an end product. The reactor circuit Cr in this embodiment, also includes the electrolyser 24, the shift reactor 126 and the separator 128.

The waste heat boiler 124 provides a thermal link between the water/steam circuit Cs and the reactor circuit Cr. Likewise, the first reactor 20 provides a thermal link between the primary energy circuit C and the reactor circuit Cr, while the water boiler 122 zo provides a thermal link between the primary energy circuit C and the water/steam circuit Cs. The electrolyser 24 forms a further heat transfer link between the water/steam circuit Cs and the reactor circuit Cr.

From this it can be seen that the primary energy circuit C transfers heat to the reformer circuit Cr via the first reactor 20 and to the water/steam circuit Cs via the boiler 122. Heat in the fluid flowing through the reformer circuit Cr is also transferred to the water/steam circuit Cs through the waste heat boiler 124.

Embodiments of the disclosed method 10e and 12e are believed to produce about 30% to 40% less carbon for the same hydrogen output. This is due to the power and thermal energy used to drive the endothermic reaction in the first reformer 20 is produced by a renewable energy source, and waste heat energy is used to augment the overall efficiency of the plant 12e. This augmentation has multiple aspects. In no specific order these aspects are:

providing the motive energy for the turbine 158 which produces electricity and powers the electrolyser 24 thereby producing additional hydrogen and oxygen used in the second reforming process;

providing heated and pressurised water for the electrolyser 24, which increases the overall efficiency of the electrolyser 24 in comparison to an electrolyser supplied with room temperature mains pressure water;

supplying steam which is heated and pressurised by energy derived solely from renewable sources for the reforming reaction in the first reactor 20; and

optionally providing electricity to power the electrical temperature control system and heater that may be incorporated in the first reactor 20.

As with previously described embodiments, the method 10e and system 12e are subject to numerous variations as to form without departing from its substantive characteristics. For example, in one variation of the CST system 40 in the energy converter 14 could be replaced by a photovoltaic array to produce electricity for heating the HTM 16 to temperature T1. A battery can also be added to store electrical energy that can be converted to heat when the sun is not shining. The electricity can power electric resistive or inductive heaters in the facility 18. In all other respects the operation of the method 10e and system 12e including the heat transfer between the HTM 16 and process fluids would be identical to that described above. Naturally power from the battery can also be used for other purposes including but not limited to:

powering utilities used in and around the plant 12, for example lights, pumps, fans; or

feed to a grid via an inverter.

In other variations the energy conversion system 14 may include one or both of a wind turbine generator and/or a geothermal heat exchanger. These may be used to offset the diurnal aspect of the solar based energy conversion systems. While there is no inherent limitation on how wind and geothermal energy may be used in the disclosed method and system it is envisaged that they would be most likely used to supplement a primary energy converter being either a CST system or a PV system. This supplementation being provided by the generation of electricity through these energy sources for storage in a battery. The stored energy can then be used in the same manner as described above in relation to the PV system.

CST system 40 that may be used in embodiments of the disclosed method and system may incorporate known commercial systems including but not limited to those manufactured by the US company Edisun Mircogrids, Inc and marketed under the brand name HELIOGEN. Depending on relatively conditions a mirror array or heliostat in a CST system may be modified to enhance performance. This could include the incorporation of mechanical structures to enable the mirrors to withstand cyclonic conditions, or automatic or autonomous mirror cleaning systems, methods and devices including robotic mirror cleaning devices. In yet a further modification mirror arrays may be formed as or otherwise supported on flexible structures that can be controllably distorted in the shape and configuration to facilitate sun tracking.

As previously mentioned in various embodiments is possible for the heat transfer medium and the heat storage medium to be one and the same, i.e. constituted by the same material. The material may be a molten salt such as calcium nitrate. Alternately the material may be in the form of a flowable particulate solid which remains in the solid phase when heated to the threshold temperature T1. Example of such material include: particulate sintered bauxite; silica; or ceramic beads or powders. These solids can be caused to flow by use of mass transfer systems such as conveyors or augers. These may be used to create for example a stream or curtain of flowing solid particle which can be heated by various means including CST. These materials and CST system that may be incorporated in embodiments of the disclosed method and plant may be sourced from for example Solex Thermal Science Inc.

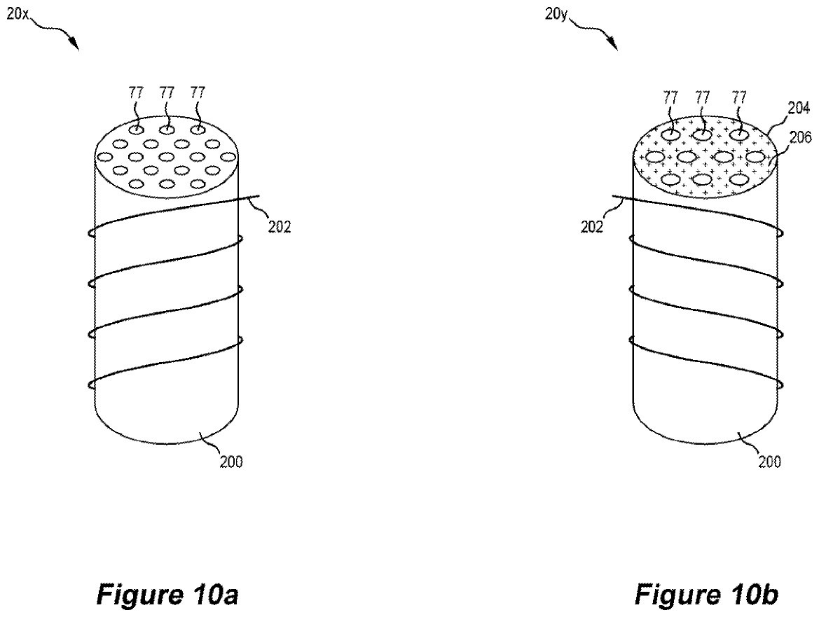

As mentioned above, heat energy for the purposes of facilitating a reaction in the reactor 20 may be achieved solely by the use of electricity by way of resistive heating or inductive heating of the reactor and/or individual conduits in the reactor through which the reactants pass. FIGS. 10a and 10b illustrate two examples of inductively heated reactors 20x and 20y respectively. The reactor 20x comprises a block 200 of electrically conductive material in which is formed a plurality of flow paths or passages 77 which may be lined with catalyst (not shown). A coil 202 for conducting an AC current wraps around the block 200. In one example the block 200 may be made from graphite. Passing an AC current through the coil 202 produces eddy currents in the block 200 generating heat. It should be appreciated that the degree of heating can be controlled by simply controlling the magnitude and frequency of the current. Also, the heat produced is uniform throughout the block 200.

The reactor 20y comprises an outer body or tube 204 and a plurality of individual conduits 77 extending through the tube 204. The volume between the inside surface of the tube 204 and the exterior surface of the conduit 77 is filled with an electrically conductive or ferromagnetic material 206, which may include, but is not limited to, graphite particles or powder. A coil 202 surrounds the outside of the tube 204. Passing an AC current through the coil 202 sets up eddy currents in the material 206 generating heat which is conducted through the conduits 77 to the catalyst in or lining the conduit 77 (not shown) and reactants flowing through the conduit 77.

With reference to the plant 10e shown in FIG. 9 the reactor 20 may be resistively or inductively heated, rather than heated by the transfer of heat from the heat storage medium 16. This would then do away with the need for the heat storage medium 16 and the associated tanks 46 and 48 as well as the CST plant 40, the boiler 122, turbine 158 and generator 160. In this example the energy conversion system 14 would comprise: renewable energy plant or transducers which generate electricity: battery storage for electricity; and associated electronics such as inverters to provide an AC current at least for inductive heating. The electrolyser 24 is powered by the electricity generated by the energy conversion system 14. In this resistively or inductively heated plant the energy transfer circuit C would be an electrical circuit for conducting electrical current rather than conducting a fluid.

It is further envisaged that an inductively heated reactor 20 may be constructed by embedding electrically conductive or ferromagnetic particles in a catalyst which either forms the conduit 77 themselves, or is provided in or as a liner for conduit 77 made from a different material. This may be achieved by way of 3D printing (e.g. 3D laser printing) catalyst structures with the electrically conductive or ferromagnetic particles.

It should also be appreciated that resistive or inductive heating can be used in other areas of the plant if required, for example for preheating the reactants, and/or to heat a heat storage medium that can be used to sully supplement heat for any area, plant or equipment of the plant.

A benefit of using this type of heating to facilitate the reaction in the reactor 20 is that it becomes possible to construct the plant and perform a method for generating the product without the need of a flowable heat transfer material. Thus, the heating can be concentrated to specific areas within the plant and the piping and use of pumps or other mass transfer apparatus for the purposes of moving the flowable heat transfer/storage material is avoided. This provides a substantial saving footprint and capital expenditure. The resistive or inductive heating can generate temperatures in excess of 2000° C. in a very targeted and confined area which also assist in avoiding or minimising the need for extensive insulation.

In the claims which follow, and in the preceding description, except where the context requires otherwise due to express language or necessary implication, the word “comprise” and variations such as “comprises” or “comprising” are used in an inclusive sense, i.e., to specify the presence of the stated features but not to preclude the presence or addition of further features in various embodiments of the system and method as disclosed herein.

What is claimed:

1. A method of reforming a hydrocarbon to produce hydrogen, methanol or other synthesis chemicals comprising:
heating a flow of a heat transfer medium by using thermal energy derived from one or more renewable energy sources to produce a flow of a superheated heat transfer medium at a threshold temperature ≥T1; and
using the flow of the superheated transfer medium to heat in parallel:
(a) a reactant feed stream including the hydrocarbon to produce a superheated reactant feed stream;
(b) a catalyst and the superheated feed stream when in a reformer reactor to produce syngas; and
(c) a heat storage medium.

2. The method of claim 1 comprising transferring heat from a flow of syngas produced in the reformer to preheat the reactant prior to producing the superheated reactant feed stream.

3. The method of claim 1, further comprising using heat from the flow of superheated heat transfer medium after producing the superheated reactant feed stream to generate electrical energy.

4. The method of claim 3, further comprising combining respective streams of the heat transfer medium used to produce the superheated reactant feed stream and the syngas to drive a turbine to produce the electrical energy.

5. The method of claim 1, further comprising using heat from a flow of the superheated heat transfer medium after producing the syngas to generate electrical energy.

6. The method of claim 5, further comprising combining respective streams of the heat transfer medium used to produce the superheated reactant feed stream and the syngas to drive a turbine to produce the electrical energy.

7. The method of claim 1, further comprising flowing a stream of the superheated heat transfer medium and the reactant through the reactor in mutually counter current directions.

8. The method of claim 1, further comprising monitoring for the temperature of the superheated heat transfer medium to fall below the threshold temperature T1 and in response transferring heat from the heat storage medium to elevate the temperature of the superheated heat transfer medium to or above the threshold temperature T1, and transferring heat from the superheated heat transfer medium at temperature T1 in parallel to the:
(a) reactant feed stream to produce a superheated reactant feed stream;
(b) the catalyst and the superheated feed stream when in a reactor to produce syngas.

9. The method of claim 8, further comprising transferring heat from the heat storage medium to elevate the temperature of the superheated heat transfer medium in addition to concurrently heating the heat transfer medium by the renewable energy source.

10. The method of claim 8, further comprising transferring heat from the heat storage medium to elevate the temperature of the superheated heat transfer medium in exclusion to heating the heat transfer medium by the renewable energy source.

11. The method of claim 1, further comprising one or more of:
using solar radiation produced by the sun as the source of renewable energy and concentrating the solar radiation to heat a heat transfer medium to produce the superheated heat transfer medium;
using solar radiation produced by the sun as the source of renewable energy, converting the solar radiation to electrical energy using one or more photovoltaic cells, and converting the electrical energy to thermal energy to heat the heat transfer medium steam
using wind as the source of renewable energy, converting the wind to electrical energy using one or more wind turbines, and using the electrical energy to heat the heat transfer medium; and
using geothermal heat as the source of renewable energy, converting the geothermal heat into electrical energy, and using the electrical energy to heat the heat transfer medium steam.

12. The method of claim 1 comprising using a combination of any two or more of:
a) a concentrated solar thermal heater;
b) one or more photovoltaic cells;
c) one or more wind turbines;
d) a geothermal heat source;
e) a wave powered generator to heat directly or indirectly the heat transfer medium.

13. The method of claim 1, wherein the hydrocarbon in methane and the reactant comprises a combination of water, carbon dioxide and the methane.

14. The method of claim 13, further comprising sourcing the methane from natural gas, coal seam gas, or decomposing organic material.

15. The method of claim 1, wherein the heat storage medium comprises a particulate solid material.

16. The method of claim 15, wherein the particulate solid material is sintered bauxite.

17. The method of claim 1 wherein the processing of the hydrocarbon produces at least one of: hydrogen, methanol, and syngas.

18. A method of processing methane to produce a target product comprising:
harnessing energy from a renewable energy source to produce a superheated heat transfer medium;
when the superheated heat transfer medium is at a threshold temperature ≥T1:
a) super heating a reactant stream which includes the methane using a first stream of the superheated heat transfer medium;
b) heating the superheated reactant stream and a catalyst when both are in a reactor using a second stream of the superheated heat transfer medium; and
c) transferring heat to a heat storage medium using a third stream of the superheated heat transfer medium; and
when the superheated heat transfer medium is below the threshold temperature T1:
d) using heat from the thermal energy storage medium to produce a feed stream of superheated transfer medium a temperature ≥T1;
e) using the superheated heat transfer medium produced by heat transfer from the heat storage medium to superheat the reactant stream, and heat the superheated reactant stream and the catalyst when in the reactor.

19. The method of claim 18, wherein harnessing energy from a renewable energy source comprises harnessing energy using any one, or a combination of any two or more of:
a) concentrated solar thermal heater;
b) one or more photovoltaic cells;
c) one or more wind turbines;
d) a geothermal heat source;
e) a wave powered generator to heat directly or indirectly the heat transfer medium to produce the superheated heat transfer medium.

20. The method of claim 18, wherein the hydrocarbon in methane and the reactant comprises a combination of water, carbon dioxide and the methane.

21. The method of claim 20, further comprising sourcing the methane from natural gas, coal seam gas, or decomposing organic material.

22. The method of claim 18, wherein the heat storage medium comprises a particulate solid material.

23. The method of claim 22, wherein the particulate solid material is sintered bauxite.

24. The method of claim 18, wherein the processing of the hydrocarbon produces at least one of: hydrogen, methanol, and syngas.

25. A plant for processing a hydrocarbon comprising:
a closed loop primary energy circuit through which a heat storage medium circulates, the primary energy circuit including an energy conversion system arranged to produce heat from one or more renewable energy sources and transfer that heat to the circulating heat storage medium;
a reformer circuit arranged to conduct one or more reactions for reforming the hydrocarbon to produce hydrogen, the primary energy circuit being arranged to transfer heat to the reformer circuit to promote the one or more reactions;
an electrolyser powered by energy sourced from the energy conversion system, the electrolysis station producing additional hydrogen to augment the hydrogen produced by the reformer circuit, and oxygen; and
a water/steam circuit thermally coupled to both the primary power circuit and the reformer circuit, the water/steam circuit arranged to produce steam and deliver the steam to:
the reformer circuit to act as a reactant with the hydrocarbon; and to the electrolyser to facilitate electrolysis of water to produce the additional hydrogen and the oxygen;
wherein the heat from the heat storage medium is transferred to the water/stream circuit.

26. The plant of claim 25, wherein oxygen produced by the electrolyser is provided to the reformer circuit to augment one of the one or more reactions.

27. The plant of claim 25, wherein the reformer circuit comprises a first reactor the first reactor being in both the primary energy circuit and the reformer circuit.

28. The plant of claim 27, wherein the first reactor has an electrical temperature control system and heater arranged to maintain a predetermined temperature range within the first reactor.

29. The plant of claim 27, wherein the reformer circuit comprises a second reactor downstream of the first reactor and the electrolyser is arranged to provide the oxygen to the second reactor, and wherein the second reactor is arranged to produce syngas which includes hydrogen.

30. The plant of claim 29, wherein the reformer circuit comprises a waste heat boiler through which the syngas flows as a heat transfer medium to transfer heat to a physically isolated fluid also flowing through the waste heat boiler.

31. The plant of claim 30, further comprising equipment downstream of the waste heat boiler and arranged to separate the hydrogen from the syngas.

32. The plant of claim 31, wherein the equipment comprises a shift reactor capable of reacting carbon monoxide and water to produce carbon dioxide and hydrogen.

33. The plant of claim 32, wherein the equipment comprises a separator downstream of the shift reactor, the separator arranged to separate carbon dioxide and hydrogen from each other.

34. The plant of claim 30, further comprising a water boiler coupled in both the primary energy circuit and the water/steam circuit wherein the heat storage medium flowing through the primary energy circuit also flows through the water boiler and transfers heat to water/steam flowing through the water boiler.

35. The plant of claim 34, wherein the waste heat boiler is in fluid communication with the water/steam circuit and is up stream of the water boiler wherein water delivered to the waste heat boiler is heated by the syngas and a fraction of the heated water flows through a conduit to the water boiler to be superheated by the heat storage medium.

36. The plant of claim 27, further comprising one or more electrically power heaters arrange to heat the heat storage medium, the first reactor, a catalyst in the first reactor, the hydrocarbon, or a combination thereof.

37. The plant of claim 36, wherein the first reactor is an inductively heated reactor comprising: one or more flow paths through which the hydrocarbon flows; a volume of electrically conductive or ferromagnetic material surrounding the flow paths; and, an electric coil wound around the flow paths.

38. The plant of claim 37, wherein the volume of electrically conductive or ferromagnetic material comprises a solid block of electrically conductive or ferromagnetic material in which a plurality of passages is formed and wherein the flow paths comprise the passages.

39. The plant of claim 38, wherein the inductively heated reactor comprises an outer body through which the one or more flow paths extend, and wherein the volume of electrically conductive or ferromagnetic material is in the form of a particulate or powder material disposed within the outer body and surrounding the flow paths.

40. The plant of claim 37, wherein the one or more flow paths comprise pipes or conduits made of a thermally conductive material.

41. The plant of claim 36, wherein the first reactor is an inductively heated reactor comprising a body containing one or more flow paths through which the hydrocarbon can flow, at least one of the flow paths flow lined with or containing a catalyst which the hydrocarbon contacts when flowing through the one or more flow paths, and wherein the catalyst includes a plurality of embedded or mixed electrically conductive or ferromagnetic material; and a coil for conducting an electric current surrounding the flow paths.
